(19) United States
(12) Patent Application Publication
(10) Pub. No.: US 2022/0125023 A1
(43) Pub. Date: Apr. 28, 2022

(54) SURVEILLANCE USING WIRELESS CONTACT LENSES ON FOWL AND LIVESTOCK

(71) Applicant: Samuel Messinger, Ramot Bet Shemish (IL)

(72) Inventor: Samuel Messinger, Ramot Bet Shemish (IL)

(21) Appl. No.: 17/080,871

(22) Filed: Oct. 27, 2020

(51) Int. Cl. A01K 45/00; A01K 29/00; G02B 1/04; H04N 7/18

(52) U.S. Cl. CPC A01K 45/00; A01K 29/005; H04W 76/10; H04N 7/18; G02B 1/043

(57) ABSTRACT

A surveillance wireless contact lens powered by body fluids for animals and fowls is disclosed using Wi-Fi, Bluetooth®, wireless local area network (WLAN), satellite and cellular tower and radio communication network. The contact lens comprises a molten material, which is injected into a mold cavity through under pressure for making the lens. The contact lens has a central optic portion with a concavo-convex shape, wherein the central optic portion having a depression at the center. A peripheral bearing portion is extended from and about the central optic portion and having an outer margin, which is placed within the limbus of an eye of the animal and/or fowl and opposite the white outer layer of the eyeball. The contact lens further comprises a processor disposed on the peripheral bearing portion, configured to control the operation of the contact lens via an electrical circuit. The electrical circuit in communication with the processor is configured to connect to electronic components include a power source, a light sensor, a communication module, and a camera.

### BACKGROUND OP THE INVENTION

### A. Technical Field

The present invention generally relates to contact lenses. More specifically, the present invention relates to a wireless surveillance contact lens for animals or livestock and fowls or birds include, hut not limited to, chickens, ducks, pheasants, turkeys and other fowls as well as mammals such as pigs, sheep, and cattle, and also horses, etc.

### B. Description of Related Art

Contact lens, or simply contacts, are thin lenses placed directly on the surface of the eyes. Contact lenses are ocular prosthetic devices used by over million people worldwide and they could be worn to correct vision or for cosmetic or therapeutic reasons. People choose to wear contact lenses for many reasons. Aesthetics and cosmetics are the main motivating factors for people who want to avoid wearing glasses or to change the appearance of their eyes. Others wear contact lenses for functional or optical reasons.

Researchers have developed a new type of robotic contact lens that can be recharged wirelessly and which could bring a wide variety of futuristic uses for contact lenses one step closer to reality. The new devious are built around a circular translucent antenna and super capacitor system that can receive continual power without needing to be plugged in to an external power source. These experimental new contact lenses will also be able to draw electricity without raising the temperature of the lens, eliminating a potential long-term cause of harm to wearers and the device itself. The lenses are completely self-enclosed and they can be maintained with standard contact solutions without any risk of degradation. The prototype contact lens uses a translucent circular antenna and double-ring supercapacitor that runs around the outer edge of the iris to make sure the users view isn't obscured. The robotic contact lens would allow its wearer to zoom in on certain objects by blinking.

In addition, the contact lens was powered by live electrodes placed around the eye on the wearer's face, and would alter its lens focal length in accordance with changes in eye muscles detected by those electrodes. The contact lens could be used not only enhance the vision of wearers people but could also monitor their eyes for potential health concerns.

Further, the University of Wisconsin is working on a lens that uses light sensors and tiny power sources to autofocus, much like a camera would, on whatever the wearer is looking at. New studies are testing lenses that could track the progress of glaucoma with tiny sensors. In future, the lenses may could measure glucose levels in patients with diabetes using eye fluids. Researchers also have developed a new type of smart contact lenses with a self-moisturizing system that could prevent dry eyes using a novel mechanism. In addition, the contact lens includes glucose sensors, wireless power transfer circuits, and display pixels to visualise sensing signals in real-time are folly integrated using transparent and stretchable nanostructures, thereby monitoring the human body in real-time.

In addition, smart contact lenses (for humans) developed Samsung record video and let the user control an electronic device, for example, a smart phone just by blinking eyes. These contact lenses feature motion sensors, which means that wearers could control devices with their eye movements and potentially give commands to their devices remotely when blinking or using their peripheral vision. The contact lenses could also beam photos and videos directly into wearers eyes.

In recent research, the contact lenses further include a wirelessly rechargeable and solid-state supercapacitor for continuous operation. Supercapacitors and different components include an antenna, a rectifier, and a light emitting diode (LED) are printed and integrated using stretchable structures to form the soft lens without obstructing vision. The device was reliable against thermal and electromagnetic radiation, with promising test results in vivo and substantial promise for smart contact lenses in the future.

A smart contact lens with an integrated camera is developed by Google. The camera would be very small and position near the edge of the contact lens so that it doesn't obscure your vision. By virtue of being part of the contact lens, the camera would naturally follow the viewer gaze and allow for a huge range of awesome applications, from the basis of a bionic eye system for blind and visually impaired people, through to early warning systems (the camera spots a hazard before your brain does), facial recognition, and superhuman powers (telescopic and infrared/night vision). Beyond the medical and consumer-oriented applications, also other possibilities, for example, if police were equipped with the contact lenses that could spot criminal faces in a crowd, or a bulge under a jacket that could be a concealed weapon. Further, the most exciting/deadly application of them all: soldiers with smart contact lenses could alert them to incoming lire, provide infrared vision that could see through smoke, and real-time range finding for mans accurate sniping.

Scientists have created a way for humans to view how the world looks like through the eyes of different animals. Researchers in Australia at the University of Queensland and a British team at the University of Exeter created a programme to shed light on the mysterious world. By analysing the properties of animals' visual systems, could model bow the world would look like through their eyes. It is hard for humans to understand what animals are really observed. Most animals have completely different visual systems to humans so for many species it is unclear bow they see complex visual information or colour patterns in nature, and how this drives their behaviour.

Currently, there are roughly 253 million people around the world living with some form of visual impairment, with the five most common conditions being: cataracts, diabetic retinopathy, macular degeneration, glaucoma and retinal detachment. Smart contact lens applications may be able to treat common eye conditions better than any conventional lens ever has. Researchers at the University of Wisconsin, for example, have invented a smart lens to treat farsightedness. Mimicking the uniquely-shaped retina of the elephant nose fish, they are designing a contact lens which autofocuses within milliseconds. Scientists at the University of Michigan are building a contact lens that gives soldiers the ability to see in the dark using thermal imaging. The technology uses a single layer of carbon atoms, called graphene, to pick up the full spectrum of light, including ultraviolet.

Unlike humans, contact lenses for animals are often used for different purposes. It has been found that behavior control of animals, such as laying chickens, ducks, pheasants, turkeys and other fowl as well as mammals such as, but not limited to, pigs, sheep, and cattle, could be achieved by applying distorting contact lenses, which reduce the clarity with which the animals can see or restrict the vision. Such contact lenses often obviate the need for sedatives or tranquilizers, which are often accompanied by deleterious effects to reduce undesired activities such as fighting with each other, picking over mash to look for coarser particles or grains, flying within their area, and nervousness.

For example, the contact lenses are applied to chickens in order to reduce aggressiveness, food wastage, expenditure of energy, noise, injuries, and resultant disease, dust and egg breakage, and also promote contentment and harmony and increase the production of eggs. These contact lenses also eliminate the necessity for debeaking chickens. Similarly, such contact lenses are useful for horses, especially during transportation in reducing tension or excitement and also maintaining a calm attitude among the horses. Similarly, two crops of lambs per year could be realized by the use of such contact lenses.

However, the existing smart contact lenses are maximum designed for humans and are opaque and brittle components have been used to enable the operation of the electronic devices, and this could block the vision and potentially damage the eyes. In addition, the use of expensive and bulky equipment to measure signals from the contact lens sensors could interfere with external activities. Henceforth, there is a need for a wireless contact lens for animals and birds in order to control behaviour and further surveillance.

In light of the above-mentioned problems, there is a need for a wireless contact lens for animals and birds such as, but not limited to, lions, cats, pigeons, chickens, and other fowl as well as mammals etc. so that could be used for further surveillance. Further, there is a need for a wireless contact lens that could be a soft, smart contact lens in which glucose sensors, light sensors, a camera, a power source, wireless power transfer circuits, and display pixels to visualize sensing signals in real time are fully integrated using transparent and stretchable nanostructures. The integration of this display into the smart lens eliminates the need for additional, bulky measurement equipment. This soft, smart contact lens could be transparent, providing distortion in order to control the behavior of the animals and/or fowls.

### SUMMARY OF THE INVENTION

The present invention generally discloses contact lenses. Further, the present invention disclose a wireless contact lens for animals or livestock and fowls or birds include, but not limited to, chickens, ducks, pheasants, turkeys and other fowls as well as mammals such as pigs, sheep, and cattle, and also horses, etc.

In one embodiment, the contact lens consists of a body of transparent material, for example, a thermoplastic resin, a medium-density or a low-density polyethylene or ethyl vinyl acetate, either clear or pigmented. In one embodiment, the contact lens could be a molten material having a central optic portion with a concavo-convex shape and a peripheral bearing portion extending from and about the central optic portion and having an outer margin which lies within the limbus of the eye. In one embodiment, the central optic portion having a depression at the center. In one embodiment, the contact lens includes slightly curved inner and outer surfaces in section to conform H to the average contour of the eye of any animal or fowl. In one embodiment, the thickness of the peripheral bearing portion preferably diminishes from its central portion to the outer margin and the inner surface is rounded adjacently the margin, as appears at to join the inner surface to the outermost bounding surface of the lens. Thereby, the contact lens is perfectly smooth at the timer surface. In one embodiment; the outer surface may have an edge, as is shown, but this is not essential, it is also free from burns and irritations.

In one embodiment, the contact lens in which the central optic portion lies its inner surface recessed to be spaced from the cornea of the eye of the fowl and provide a chamber. When applied to the fowl, the nictitating membrane, which is situated beneath the peripheral bearing portion could enter into this chamber. The outer eyelids overlie the peripheral bearing portion. The contact lens is preferably of circular outline. This has the advantage of facilitating installation of the contact lens and of reducing the tendency for displacement of the lens from the eye of the fowl. In one embodiment, the user could insert the contact lens deeply under the lower and outer eyelid by fingers and then pulling back the upper eyelid, thereby permitting the contact lens to fell into place. The contact lens is then moved to upwards for positioning at the center. In one embodiment, a vacuum applicator could be used for holding the contact lens and it is also not required.

In one embodiment, the timer surface of the central portion is concave, preferably with a radius of curvature different from, e.g. larger than, that of the outer surface to produce distortion. Further, the inner surface is recessed with reaped to the peripheral bearing portion so as to lie in spaced relation to the cornea of the eye, thereby providing a chamber. Further shown are the following parts of the eye: a sclera, a nictitating membrane, the outer eyelids, a scleral ring, an anterior chamber and lens. The peripheral bearing portion overlies the sclera or scleral ring and, when applied to the fowl, also the nictitating membrane. This membrane can enter into the chamber when the fowl blinks its inner eyelid or nictitating membrane. The outer eyelids and overlie the peripheral bearing portion.

In one embodiment, the central part of the outer surface has a depression, wherein the depression has a diameter, which is a minor fraction of the diameter of the central optic portion, preferably less than one-fifth of this diameter, for example, one-tenth of this diameter. In one embodiment, the depression may be of any shape but is preferably round and concave in cross section. This depression is introduced and added distortion into the lens at the optical axis. In one embodiment, the contact lens is formed by using a molding process, in which the molten material is injected into the mold through a gate which is a fine passage for passing through a protuberance in the mold surface. In one embodiment, the contact lens could be used for animals and fowls or birds include, but not limited to, chickens, ducks, pheasants, turkeys and other fowls as well as mammals such as pigs, sheep, and cattle, and also horses, etc.

In some embodiments, the contact lens could be a soft, smart contact lens in which glucose sensors, wireless power transfer circuits, and display pixels to visualize sensing signals in real time are folly integrated using transparent and stretchable nanostructures. The integration of this display into the smart lens eliminates the need for additional, bulky measurement equipment. This soft, smart contact lens could be transparent, providing distortion in older to control the behavior of the animals and/or fowls.

In a one embodiment, the contact lens further comprises tiny power sources to autofocus, much like a camera on whatever the animal or fowl is looking at. In one embodiment, the contact lens could track the progress of glaucoma using tiny sensors. In one embodiment, the contact lens is further configured to measure glucose levels in eye fluids. In one embodiment, the contact lens is further configured to prevent dry eyes using a self-moisturizing system, which maintains a layer of fluid between the contact lens and the eye using a novel mechanism. In one embodiment, the contact lens is further configured to monitor the eyes for potential health concerns. In some embodiment, the contact lens could be used for humans.

In one embodiment, the contact lens further comprises a processor, which is securely disposed on the peripheral bearing portion. The processor is configured to control the operation of the contact lens via an electrical circuit. In one embodiment, the electrical circuit could be an integrated electrical circuit. In one embodiment, the electrical circuit in communication with the processor is configured to connect to electronic components include a communication module, at least one power source, and at least one light sensor.

In one embodiment, the power source in communication with the electrical circuit is configured to supply power to the electrical circuit and other electronic components. In one embodiment, the power source could be, but not limited to, a rechargeable bio fuel micro battery such as U.S. Pat. No. 10,340,546 Messinger/Kahn. In another embodiment, the power source could be, but not limited to, a super capacitor. In one embodiment, the power source is configured to recharge using body fluids. In one embodiment, the power source can be charged wirelessly via an antenna and without raising the temperature of the contact lens and also eliminating a potential long-term cause of harm to the eyes and the contact lens itself. In one embodiment, the antenna is configured to receive continual power without needing to be plugged in to an external power source.

In one embodiment the light sensor is connected to the electrical circuit and configured to determine amount of ambient light in the environment of the animal and/or fowls. In one embodiment, the contact lens further comprises at least one camera, wherein the camera in communication with the processor is configured to capture the environment of the animal and/or the fowl.

In one embodiment, the communication module is configured to wirelessly transmit signals and data from the contact lens and to an external communication source and vice versa from the external communication source fat controlling the contact lens. In one embodiment, the communication module is further configured to receive and transmit signals from an external device or a communication source to the power source via the antenna. In one embodiment, the communication module is wirelessly connected to the external device or communication source via a network. In one embodiment, the network is at least any one of, but not limited to, Wi-Fi, Bluetooth®, a wireless local area network (WLAN), and a radio communication network.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when reed in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows a perspective view of a wireless contact lens applied to a fowl, for example, a chicken by a user in an embodiment of the present invention.

FIG. 2 shows a side perspective view of the wireless contact lens applied to the fowl in one embodiment of the present invention.

FIG. 3 draws a schematic view of the wireless contact lens in one embodiment of the present invention.

FIG. 4 shows a schematic view of the contact lens configured to wirelessly communicate to a ground station via a network, in one embodiment of the present invention.

FIG. 5 shows a schematic view of the ground station receiving signals from the contact lens via a transceiver for monitoring the bird and eye view using a computing device in an exemplary embodiment of the present invention.

FIG. 6 shows a sectional view of a rechargeable bio Aral micro battery in an exemplary embodiment of the present invention.

FIG. 7 shows a sectional view of a two-part mold with an upper section and a lower section used for molding the wireless contact lens in one embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

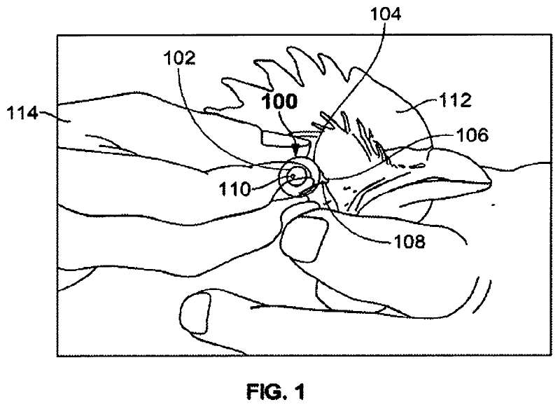

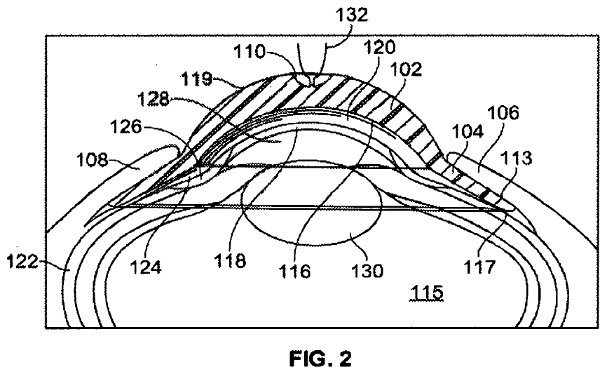

Referring to FIGS. 1-2, a wireless contact lens 100 applied to a fowl 112, far example, a chicken by a user 114 in one embodiment is disclosed. In one embodiment, the contact lens 100 consists of a body of transparent material, for example, a thermoplastic resin, a medium-density or a low-density polyethylene or ethyl vinyl acetate, either clear or pigmented. In one embodiment, the contact lens 100 could be a molten material having a central optic portion 102 with a concavo-convex shape and a peripheral bearing portion 104 extending from and about the central optic portion 102 and having an outer margin 113 which lies within the limbus of the eye 115. In one embodiment, the central optic portion 102 having a depression 110 at the center. In one embodiment, the contact lens 100 includes slightly curved inner and outer surfaces in section to conform it to the average contour of the eye of any animal or fowl. In one embodiment, the thickness of the peripheral bearing portion 104 preferably diminishes from its central portion to the outer margin 113 and the inner surface is rounded adjacently the margin, as appears at 117 to join the inner surface 116 to the outermost bounding surface of the lens 130. Thereby, the contact lens 100 is perfectly smooth at the inner surface 116. In one embodiment, the outer surface 119 may have an edge, as is shown, but this is not essential, it is also free from burrs and irritations.

In one embodiment, the contact lens 100 in which the central optic portion 102 has its inner surface recessed to be spaced from the cornea 118 of the aye 115 of the fowl 112 and provide a chamber. When applied to the fowl 112, the nictitating membrane 124, which is situated beneath the peripheral bearing portion 104 could enter into this chamber. The outer eyelids 106 and 108 overlie the peripheral bearing portion 104. The contact lens 100 is preferably of circular outline. This has the advantage of facilitating installation of the contact lens 100 and of reducing the tendency for displacement of the lens 130 from the eye 115 of the fowl 112. In one embodiment, the user 114 could insert the contact lens 100 deeply under the lower and outer eyelid by fingers and dun pulling back the upper eyelid, thereby permitting the contact lens 100 to fall into place. The contact lens 100 is then moved to upwards for positioning at the center. In one embodiment, a vacuum applicator could be used for holding the contact lens 100 lens and it is also not required.

In one embodiment, the recessed inner surface lid of the central portion is concave, preferably with a radius of curvature different from, e.g. larger than, that of the outer surface 119 to produce distortion. Further, the inner surface lid is recessed with respect to the peripheral bearing portion 104 so as to lie in spaced relation to the cornea 118 of the eye 115, thereby providing a chamber 120. Further shown are the following parts of the eye: a sclera 122, a nictitating membrane 124 (also called the third or inner eyelid), the outer eyelids 106 and 108, a scleral ring, an anterior chamber 128 and lens 130. The peripheral bearing portion 104 overlies the sclera 122 or scleral ring 126 (herein for convenience collectively called the sclera) and, when applied to the fowl 112, also the nictitating membrane 124. This membrane can enter into the chamber 120 when the fowl 112 blinks its inner eyelid or nictitating membrane 124. The outer eyelids 106 and 108 overlie the peripheral bearing portion 104.

In one embodiment, the central optic portion 102 of the outer surface 119 has a depression 110, wherein the depression 110 has a diameter, which is a minor fraction of the diameter of the central optic portion 102, preferably less than one-fifth of this diameter, for example, one-tenth of this diameter. In one embodiment, the depression 110 may be of any shape but is preferably round and concave in cross section as shown in FIG. 2. This depression 110 is introduced and added distortion into the lens 130 at the optical axis. In one embodiment, the contact lens 100 is formed by using a molding process, in which the molten material is injected into the mold through a gate 132 which is a fine passage for passing through a protuberance in the mold surface. In one embodiment the contact lens 100 could be used for animals and fowls or birds include, but not limited to, chickens, ducks, pheasants, turkeys and other fowls as well as mammals such as pigs, sheep, and cattle, and also horses.

In some embodiments, the contact lens 100 could be a soft, smart contact lens in which glucose sensors, wireless power transfer circuits, and display pixels to visualize sensing signals in real time are folly integrated using transparent end stretchable nanostructures. The integration of this display into the smart lens eliminates the need for additional, bulky measurement equipment. This soft, smart contact lens could be transparent, providing distortion in order to control the behavior of the animate and/or fowls.

In one embodiment, the contact lens 100 further comprises tiny power sources to autofocus, much like a camera on whatever the animal or fowl is looking at. In one embodiment, the contact lens 100 could track the progress of glaucoma using tiny sensors. In one embodiment the contact lens 100 is further configured to measure glucose levels in eye fluids. In one embodiment, the contact lens 100 is further configured to prevent dry eyes using a self-moisturizing system, which maintains a layer of fluid between the contact lens 100 and the eye using a novel mechanism. In one embodiment, the contact lens 100 is further configured to monitor the eyes for potential health concerns. In some embodiment, the contact lens 100 could be used for humans.

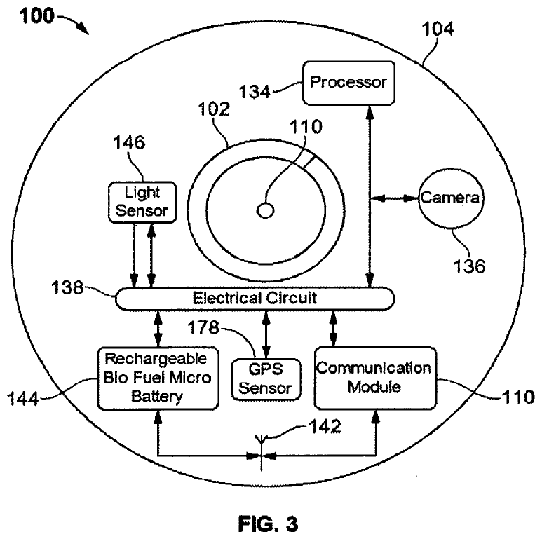

Referring to FIG. 3, a schematic view of the contact lens 100 in one embodiment is disclosed. In one embodiment, the contact lens 100 further comprise a processor 134, which is securely disposed on the peripheral bearing portion 104. The processor 134 is configured to control the operation of the contact lens 100 via an electrical circuit 130. In one embodiment, the electrical circuit 138 could be an integrated electrical circuit. In one embodiment the electrical circuit 138 in communication with the processor 134 is configured to connect to electronic components include a communication module 140, at least one power source 144, and at least one light sensor 146. In one embodiment, the power source 144 in communication with the electrical circuit 138 is configured to supply power to the electrical circuit 138 and other electronic components. In one embodiment, the power source 144 could be, but not limited to, a rechargeable bio fuel micro battery. In another embodiment, the power source 144 could be, but not limited to, a super capacitor. In one embodiment, the power source 144 is configured to recharge using body fluids. In one embodiment, the power source 144 can be charged wirelessly via an antenna 142 and without raising the temperature of the contact lens 180 and also eliminating a potential lung-term cause of harm to the eyes and the contact lens 180 itself. In one embodiment, the antenna 142 is configured to receive continual power without needing to be plugged in to an external power source.

In one embodiment, the tight sensor 140 is connected to the electrical circuit 138 and configured to determine amount of ambient light in the environment of the animal and/or fowls. In one embodiment, the contact lens 180 further comprises at least one camera 130, wherein the camera 136 in communication with the processor 134 is configured to capture the environment of the animal and/or the fowl.

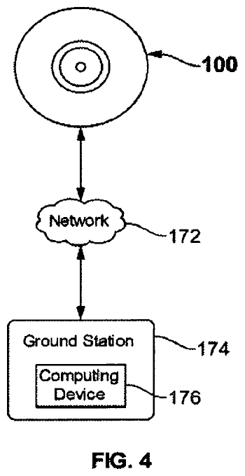

In one embodiment, the communication module 140 is configured to wirelessly transmit signals and data from the contact lens 100 and to an external communication source and vice versa from the external communication source for controlling the contact lens 100. In one embodiment, the communication module 140 in further configured to receive and transmit signals from an external device or a communication source to the power source 144 via the antenna 142. In one embodiment, the communication module 140 is wirelessly connected to the external device or communication source via a network 172 (shown in FIG. 4). In one embodiment, the network is at least any one of but not limited to, Wi-Fi, Bluetooth®, a wireless local area network (WLAN), and a radio communication network. In one embodiment, the contact lens 180 further comprises a global positioning system (GPS) sensor to determine GPS coordinates of the fowl or animal. The GPS coordinates could be transferred to the ground station 174 (shown in FIG. 4) to determine the location of the fowl or animal.

Referring to FIG. 4, the contact lens 100 configured to wirelessly communicate to a ground station 174 via a network 172 in one embodiment is disclosed. Another embodiment is satellite (not shown) and cellular tower linkage (not shown). In one embodiment the contact lens 100 is configured to transfer data and signals to and from the ground station 174. (n one embodiment, the data includes images and videos that ere captured by the camera 136, which is installed on the camera lens 100, thereby efficiently collecting the data for surveillance using the contact lens 100 on the fowls and animals or livestock. In one embodiment, the network 172 is at least any one of, but not limited to, Wi-Fi, Bluetooth®, a wireless local area network (WLAN), and a radio communication network. In one embodiment, the ground station 174 further comprises a computing device 176, wherein the competing device 176 is configured to send signals to the contact lens 160 and receive the data from the contact lens 100 and vice versa. In one embodiment, the computing device 176 is at least any one or a combination of, but not limited to, a computer, a laptop, a smartphone, a remote device, a personal digital assistant (PDA), a mobile phone, and a tablet, hi one embodiment, the computing device 176 comprises a processor, a memory, a communication module, and etc. The memory could include computer storage media in the form of volatile and/or nonvolatile memory such as read only memory (ROM) and/or random-access memory (RAM).

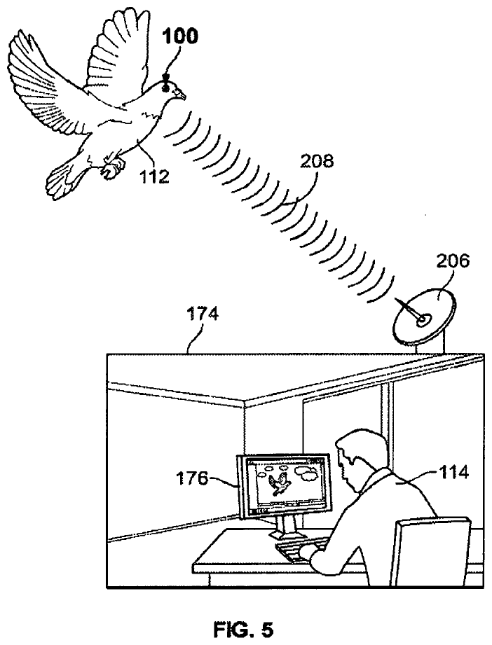

Referring to FIG. 5, a schematic view of the ground station 174 receiving signals 200 from the contact lens 100 via a transceiver 206 for monitoring the bird 112 and eye view using the computing device 176 in one embodiment is disclosed. In ono embodiment, the contact lens 100 is configured to send signals 200 to the ground station or satellite (not shown) or cellular tower (not shown) 174. In one embodiment, the ground station 174 further comprises a transceiver 206. The transceiver 206 is configured to transfer and receive (vice versa) signals 200 from the contact lens 100 via a network 172 (shown in FIG. 4) such as, but not limited to, Wi-Fi, Bluetooth®, a wireless local area network (WLAN), and a radio communication network. In one embodiment, the user could monitor the bird 112 and eye view of the bird 112 using the computing device 176, for example, a screen or a display.

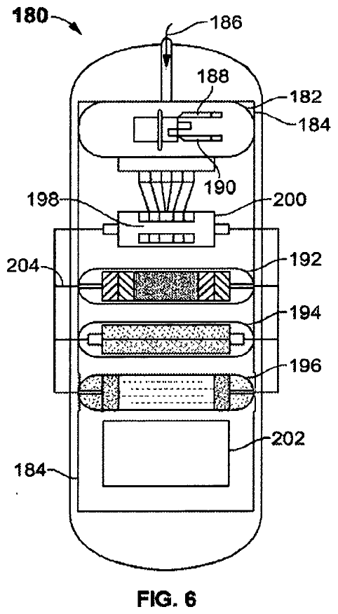

Referring to FIG. 6, a rechargeable bio fuel micro battery such as in U.S. Pat. No. 10,340,546 Messinger/Kahn 180 in one embodiment is disclosed. In one embodiment, the contact lens 100 could be provided with the bio-fuel micro battery 180. In some embodiments, the micro battery 180 could be safely placed inside the fowl or animal. In one embodiment, the bio-fuel micro battery 180 is configured supply electrical power to the contact lens 100. In one embodiment, the bio-fuel micro battery 180 is made of biocompatible and inert materials in order to prevent or reduce any adverse immunological response to micro battery. In some preferred embodiments, the bio-fuel micro battery 180 is one of voltaic cells that generates electricity on the standard principle of biocompatible compartment 182, which is also referred to as a primary compartment. The biocompatible compartment 182 is configured to store a chemical substance, which may include but is not limited to: an acidic solution or sugar solution mainly including hydrochloric acid, small quantities of potassium chloride and sodium chloride, or sugar/glucose and the like used as the electrolyte.

In some embodiments, the biocompatible compartment 182 is filled with acid, and a refill electrolyte is supplied through bio membrane 184, which is coupled with a balloon nipple 186 using an external injection device. In some embodiments, the biocompatible compartment 182 is refueled by directly swallowing biological acid jukes, which may include but are not limited to: lemon juice, orange juice, pineapple juice, and any other sour juice and the like. The re tillable balloon nipple 186 is made of a biocompatible silken rubber in order to inject the solution externally, and the repeated injections of silicon rubber balloons do not hamper the body. In some other embodiments, the biocompatible compartment 182 is refueled with the biological acid jokes through intravenous therapy by infusing the predefined chemical substance into body. Also, in some embodiments, the biocompatible compartment 182 is refueled with the biological acid juices fay using at least one akin patch, a foot bath and the like processes to infuse predefined chemical substances into the body that travel to the micro battery 180, and enter the biocompatible compartment 182, e.g. via diffusing across the nipple 186.

Additionally, bio-membrane 184 is configured to diffuse at least one bio-fluid across the anode electrode and the cathode electrode (188 and 190) to generate electron follow far recharging the micro battery and/or for supplying power to a connected bio-medical implanted device. In one embodiment, bio-membrane 184 comprises: a biocompatible compartment 182 storing at least one of a chemical substance configured to generate electricity to power the bio-medical implanted device (not shown); and one or more bio-fuel compartments 192, 194, and 196 configured to store at least one biofuel for generating on electrolyte to create a conductive path for electrons emitted by the electrodes (188 and 190). In one embodiment, the micro battery 180 further comprises a multipurpose compartment 202.

In one embodiment, the micro battery 180 further comprises at least one microprocessor 200 in communication with the biocompatible compartment 182 through a plurality of connectors 198 that interface with the bio-fuel compartments (192, 194, and 196) to control the communication between the computing device 176 (shown in FIG. 4) and the contact lens 190.

In some embodiments, the bio-fuel compartment 192 is included in the bio-membrane to recharge the micro battery 189 by itself from the blood stream. In this current process, the body heat is used as a power source for the micro battery 180 and further used as a power supply for the entire body. As the body temperature differs in different places of the body, the biocompatible compartment 182 of the micro battery 188 provides a power backup to maintain the constant power supply through the instruction of the processor 209 controlled by the computing device 176 via a software application.

In biofuel compartments, the power is generated by causing a chemical reaction at a controlled rate and by burning blood chemicals from blood cells of the animal or fowl body. The electrodes 294 of the blood fool compartments (192, 194, and 196) are coupled with flesh to create a conductive path for the electrons emitted by the electrodes 204 for operating the contact lens 190.

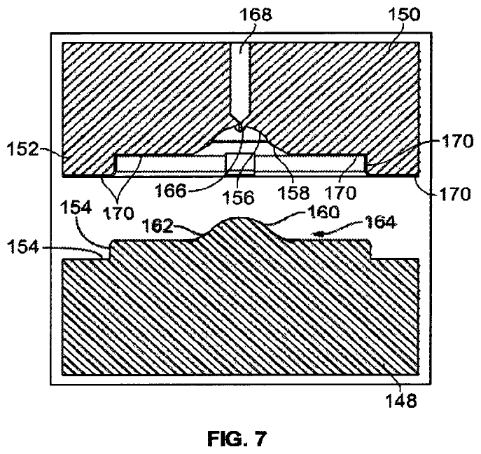

Referring to FIG. 7, a sectional view of a two-part mold with an upper section 148 and a lower section ISO used for molding the contact lens 109 in one embodiment is disclosed. In one embodiment, the upper section 148 and lower section ISO include guides such as an annular projection 152 on the upper section 148 and an annular recess 154 on the lower section 150, for insuring alignment. In one embodiment, the molding cavity is defined by molding surfaces (156 and 158) in the upper section ISO for the upper surfaces of the central optic portion 102 and peripheral bearing portion 104, respectively, and corresponding surfaces (169 and 162) on the lower section 148 for molding the lower surfaces of these lens portions.

The latter is rounded at its outer edge, as appears at 164, to produce a rounded margin in the lens. The surface 156 has a downward protuberance 166 through which extends a vertical passage or gate 168 for the supply of molten material. This gate 168 is shown to be open to the top of the mold section, but it should be understood that in molds having several molding cavities for forming a plurality of lenses simultaneously, the gates to these cavities would be connected by runners, as is understood in the molding art. The mold is further provided with means for exhausting air, e.g. four radial vent channels 179 milled in the tapper section ISO to a very shallow depth, for example, 0.0007 inches. It is evident that air could be vented in other ways, for example, about a vertically movable stripper pin, which may be provided at the center of the lower section 148 to eject the hardened lens from the mold. Such a stripper pin is well known in the art.

In molding, the material, for example, a thermoplastic resin is injected into the mold cavity through the gate 108 underpressure and displaced air flows out through a vent channel 170. This vent channel 170 is so fine that allows gas flow through it, thereby preventing flashing during the process. The mold sections (148 and ISO) are separated for removing the contact lens 100 after hardened, and breaking off of the ligament extending from the central depression 156 to the gate can be affected. In one embodiment, the central depression 110 is irregularly or unevenly molded by the protuberance 166 to introduce blurring at the optical axis. This is desirable in a distorting lens. The contact lens 100 is perfectly smooth save at the center of the outer surface.

In an exemplary embodiment, the diameter of the central optic portion 102 of the contact lens 100 for a chicken is about, but not limited to, 0.36 inches and diameter of the lens is about, but not limited to, 0.585″. In one embodiment, the maximum thickness of the central optic portion 102 is about, but not limited, 0.035″, radius of the outer surface of the central optic portion 102 is about, but not limited to, 0.200″, and the radius of the inner surface of the central optic portion 102 is about, but not limited to, 0.0185″. In an exemplary embodiment; the inclination of peripheral bearing portion 104 and angle from the upper surface to a base plane is about, but not limited to, 34 degrees. In one embodiment, the thickness of the peripheral bearing portion 104 adjacent to the central optic portion 102 is about, but not limited to, 0.025″ and the thickness at the margin is about, but not limited to, 0.015″. In one embodiment, the radius of the curvature at the outer margin is about, but not limited to, 0.031″ and the depth of the central depression is about, but not limited to, 0.015″. In one embodiment, the maximum diameter of the central depression is about, but not limited to, 0.031″.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A wireless contact lens for animals and fowls, comprising:
a molten material having a central optic portion with a concavo-convex shape, wherein the central optic portion having a depression at the center and a recessed inner surface;
a peripheral bearing portion extending from and about the central optic portion, wherein the peripheral bearing portion having an outer margin, which is placed within the limbus of an eye of the animal and/or fowl and opposite the white outer layer of the eyeball; and wherein a thickness of the peripheral bearing portion diminishes from its central portion to the outer margin and the inner surface is rounded adjacently the outer margin to loin the Inner surface to an outermost bounding surface of the wireless contact lens;
a processor disposed on the peripheral bearing portion, wherein the processor is configured to control the operation of the wireless contact lens via an electrical circuit;
a glucose sensors, wireless power transfer circuits and a display pixels to visualize a sensing signals are integrated within wireless contact lens using transparent and stretchable nanostructures; and
a self-moisturizing system configured to prevent dry eyes by maintaining a layer of fluid between the wireless contact lens and the eye of the animal;
wherein the electrical circuit in communication with the processor is configured to connect to electronic components include at least one power source, at least one light sensor, and a communication module, and
wherein the power source in communication with the electrical circuit is configured to supply power to the electrical circuit and the electronic components, wherein the wireless contact lens having slightly curved inner and outer surfaces in section to conform it to the average contour of the eye of the animal and/or fowl;
wherein the glucose sensor is configured to monitor a glucose level in eye fluids; and
wherein the communication module is further configured to allow a user to monitor an eye view of the animals and fowls using an external user computing device.

2. The wireless contact lens of claim 1, wherein the recessed inner surface is spaced from the cornea of the eye of the animal and/or fowl to form a chamber for receiving a nictitating membrane of the eye.

3. The wireless contact lens of claim 1, wherein the depression having a concave shape and a diameter less than one-fifth of the diameter of the central optic portion.

4. (canceled)

5. The wireless contact lens of claim 1, wherein the molten material is at least any one of a thermoplastic resin and a polymeric resin.

6. The wireless contact lens of claim 1, wherein the power source is a rechargeable bio fuel micro battery.

7. The wireless contact lens of claim 1, wherein the power source is configured to recharge using body fluids.

8. The wireless contact lens of claim 1, wherein the light sensor is connected to the electrical circuit and configured to determine amount of ambient light in the environment of the animal and/or fowl.

9. The wireless contact lens of claim 1, further comprises at least one camera, wherein the camera in communication with the processor is configured to capture the environment of the animal.

10. The wireless contact lens of claim 1, wherein the communication module is configured to wirelessly transmit signals and data from the wireless contact lens and to an external communication source and vice versa from the external communication source for controlling the wireless contact lens such as satellite and/or cellular tower.

11. The wireless contact lens of claim 1, wherein the communication module is further configured to receive and transmit signals from the external user computing device or a communication source to the power source via an antenna.

12. The wireless contact lens of claim 1, wherein the communication module is wirelessly connected to the external user computing device or communication source via a network.

13. The wireless contact lens of claim 12, wherein the network is at least any one of Wi-Fi, Bluetooth®, a wireless local area network (WLAN), and a radio communication network.

14. The wireless contact lens of claim 1, is made of a transparent material, which is injected into a mold cavity through under pressure for making the wireless contact lens.

15. The wireless contact lens of claim 1, wherein the animals and birds include chickens, ducks, pheasants, turkeys and other fowls as well as mammals such as pigs, sheep, and cattle, and also horses.

16. A wireless contact lens for animals and fowls, comprising:
transparent material injected into a mold cavity through under pressure to form a central optic portion with a concavo-convex shape, wherein the central optic portion having a depression at the center and a recessed inner surface,
wherein the depression having a concave shape and a diameter less than one-fifth of the diameter of the central optic portion;
a peripheral bearing portion extending from and about the central optic portion, wherein the peripheral bearing portion having an outer margin, which is placed within the limbus of an eye of the animal and/or fowl and opposite the white outer layer of the eyeball;
wherein the peripheral bearing portion slopes away from the outer surface of the central optic portion and said peripheral bearing portion has a thickness which diminishes from said central optic portion to the margin and the Inner surface Is rounded adjacently the Qatar margin to lain the toner surface to an outermost bounding surface of the wireless contact lens;
a processor secured on the peripheral bearing portion, wherein the processor is configured to control the operation of the wireless contact lens using an electrical circuit;
a glucose sensors, wireless power transfer circuits and a display pixels to visualize a sensing signals are integrated within wireless contact lens using transparent and stretchable nanostructures; and
a self-moisturizing system configured to prevent dry eyes by maintaining a layer of fluid between the wireless contact lens and the eye of the animal;
wherein the electrical circuit in communication with the processor is configured to connect to electronic components include at least one a rechargeable bio fuel micro battery, at least one light sensor, and a communication module,
wherein the light sensor is connected to the electrical circuit and configured to determine amount of ambient light in the environment of the animal;
wherein the power source in communication with the electrical circuit is configured to supply power to the electrical circuit and the electronic components, and
at least one camera in communication with the processor, configured to capture the environment of the animal,
wherein the wireless contact lens having slightly curved inner and outer surfaces in section to conform it to the average contour of the eye of the animal and/or fowl; and
wherein the glucose sensor is configured to monitor a glucose level in eye fluids; and
wherein the communication module is further configured to allow a user to monitor an eye view of the animals and fowls mine an external user computing device.

17. The wireless contact lens of claim 16, wherein the recessed inner surface is spaced from the cornea of the eye of the animal and/or fowl to form a chamber for receiving a nictitating membrane of the eye.

18. The wireless contact lens of claim 16, wherein the transparent material is at least any one of a molten material, a thermoplastic resin, and a polymeric resin.

19. The wireless contact lens of claim 16, wherein the communication module is configured to wirelessly transmit signals and data from the wireless contact lens and to an external communication source and vice versa from the external communication source for controlling the wireless contact lens, wherein the communication module is further configured to receive and transmit signals from the external user computing device or a communication source to the power source via an antenna.

20. The wireless contact lens of claim 16, wherein the communication module is wirelessly connected to the external device or communication source via a network, wherein the network is at least any one of Wi-Fi, Bluetooth®, a wireless local area network (WLAN), and a radio communication network.
